(19) United States
(12) Patent Application Publication
(10) Pub. No.: US 2019/0365287 A1
(43) Pub. Date: Dec. 5, 2019

(54) APPARATUS AND METHOD FOR GAIT TYPE CLASSIFICATION USING PRESSURE SENSOR OF SMART INSOLE

(71) Applicant: Industry-Academic Cooperation Foundation, Dankook University, Yongin-si (KR)

(72) Inventors: SANG-IL CHOI, Seoul (KR); Wooduk SEO, Daegu (KR); Sungsin LEE, Suwon-si, Gyeonggi-do (KR)

(73) Assignee: Industry-Academic Cooperation Foundation, Dankook University, Yongin-si, Gyeonggi-do (KR)

(21) Appl. No.: 16/191,616

(22) Filed: Nov. 15, 2018

(30) Foreign Application Priority Data
May 30, 2018 (KR) 10-2018-0061894
May 31, 2018 (KR) 10-2018-0062806

(51) Int. Cl. A61B 5/11; A61B 5/103; A61B 5/00; A43B 17/00; A61B 5/0488

(52) U.S. Cl. CPC A61B 5/112; A61B 5/1038; A61B 5/6807; A43B 17/00; A43B 3/0005; A61B 5/4082; A61B 2562/227; A61B 2562/0247; A61B 5/0022; A61B 5/0488

(57) ABSTRACT

Disclosed are an apparatus and a method for gait type classification using a pressure sensor of a smart insole, which are capable of classifying pieces of gait data having various variances using only a pressure sensor. The apparatus for gait type classification includes a gait data measuring part configured to measure pieces of gait data using a pressure sensor, a pre-processor configured to define a section of a unit step in all the pieces of gait data, divide the pieces of gait data for each unit step, and normalize the pieces of divided gait data to equalize lengths of the pieces of divided gait data, and a feature extractor configured to extract features suitable for gait type classification from the pieces of pre-processed gait data, and a gait type classifier configured to receive the extracted features as an input and determine and classify a final gait type.

### CROSS-REFERENCE TO PRIOR APPLICATIONS

This application claims priority from and the benefit of Korean Patent Application Nos. 10-2018-0061894 filed on May 30, 2018 and 10-2018-0062806 filed on May 31, 2018, which are all hereby incorporated by reference in their entirety.

### BACKGROUND

### 1. Field of the Invention

The present invention relates to gait type analysis, and more particularly, to an apparatus and a method for gait type classification using a pressure sensor of a smart insole, which are capable of classifying pieces of gait data having various variations using only a pressure sensor.

### 2. Discussion of Related Art

A gait is one of the typical behaviors of human beings, and gait type analysis may be applied in many application fields such as bioengineering, rehabilitation medicine, healthcare, and the like.

As a detailed field of the gait type analysis, gait type classification is being studied for diagnosis of Parkinson's disease, sports analysis, and development of a gait assistance device for the elderly on the basis of pieces of gait data acquired by sensors.

A gait type has characteristics which are easily influenced by factors such as a physical difference and a speed due to a body characteristic and a terrain difference, and these characteristics make a large variation in the same gait type and adversely affect performance of the gait type classification.

For example, even in the same gait motion, there are differences in gait type when walking on flat ground and a hill, and these variations make it difficult to extract features so as to classify the gait type which is a “step.”

A gait type classification system is configured with a sensor module for acquiring pieces of sensor data and an application module for calculating a classification result on the basis of the pieces of acquired sensor data. Sensors used in the gait type classification mainly include video sensors, electromyographic (EMG) sensors, plantar pressure sensors, accelerometer sensors, and gyroscope sensors.

However, most of these sensors have limitations in measuring pieces of gait data in only a limited environment due to restrictions, such as a sensor size and inconvenience of installation.

Recently, the development of a wearable sensor technique has led to weight reduction and simplification of equipment which is used to measure pieces of gait data.

Research on gait type classification is actively being carried out for alleviating restrictions of a place and an action due to the above-described factors, and research on gait type classification using an accelerometer sensor of the smartphone, and a smart watch, and a gyroscope sensor is mainly being carried out.

Recently, a smart insole with pressure sensors has been developed and research for gait type classification has been attempted using information on a surface of a sole in addition to accelerometer sensors, and gyroscope sensors.

An application module may be broadly divided into three operations, i.e., a pre-processing operation, a feature extraction operation, and a classification operation.

In the pre-processing operation, noise is removed from pieces of collected data and the pieces of collected data are converted into a form suitable for analysis. To this end, noise reduction filters such as a low-pass filter, a moving average filter, and a multi-level wavelet decomposition filter are used, and methods such as a zero-crossing detection method and a sliding window are used to define a step unit.

The feature extraction operation extracts features, which may be easily classified, from pieces of data, and various types of linear discriminant analysis methods and a machine learning method including a neural network may be used.

In the classification operation, a K-nearest neighbor (NN) classifier and a support vector machine may be used.

Methods for gait type classification of a related art will be described below.

FIG. 1 is a block diagram of an apparatus for gait type classification using a pressure sensor according to the related art, and FIG. 2 is a block diagram of an apparatus for gait type classification using an accelerometer sensor and a gyroscope sensor according to the related art.

FIG. 1 illustrates a classifier for classifying gait types on stairs and on flat ground by applying kernel principal component analysis (PCA) and a support vector machine (SVM) to pieces of gait data acquired from pressure sensors of a smart insole.

However, since the PCA reflects undesired variances by projecting the pieces of gait data in a direction in which variance of a covariance matrix for the pieces of gait data is maximized, the PCA has a limitation in that it is not suitable for classification of pieces of gait data having various variances.

FIG. 2 illustrates a classifier for classifying gait types on flat ground and on stairs using features to which wavelet decomposition is applied after acquiring pieces of gait data from an accelerometer sensor or a gyroscope sensor, which are attached to a shoe.

The classifier classifies a status of a gait on flat ground, stairs, and a hill using descriptive statistics, such as an average, a maximum, a minimum, a correlation coefficient of the pieces of gait data acquired from a pressure sensor, an accelerometer sensor, and a gyroscope sensor of a smart insole, as features.

While accuracy of classification may be increased when all of the pressure sensor, the accelerometer sensor, and the gyroscope sensor are used, there is a disadvantage in that costs and computation times increase with the use of continuous values.

Further, in order to use the descriptive statistics as features, a sufficient volume of the pieces of gait data should be accumulated, and this also becomes a cause of increasing a time required for gait type classification.

Specifically, although a high classification rate is exhibited for the gait type measured on the flat ground and on the stairs, a great deal of calculation is required for peak detection, and in the case of the gait type having a large time variation, stable peak detection is difficult such that there is a limitation in which the descriptive statistics cannot be applied to various gait type classification.

Therefore, it is required to develop a technique for new gait type classification capable of obtaining a robust classification result with respect to variances of piece of gait data which will occur according to a gait environment with a simplified hardware structure and a small calculation amount.

### PRIOR ART DOCUMENT

### Patent Document

(Patent Document 1) Korean Patent Laid-Open Application No. 10-2017-0110981
(Patent Document 2) Korean Registered Patent No. 10-1583369
(Patent Document 3) Korean Patent Laid-Open Application No. 10-2013-0127517

### SUMMARY

The present invention is directed to an apparatus and a method for gait type classification using a pressure sensor of a smart insole, which are capable of classifying pieces of gait data having various variances using only a pressure sensor.

Further, the present invention is directed to an apparatus and a method for gait type classification using a pressure sensor of a smart insole, which are capable of defining a section corresponding to one step in pieces of entire data by detecting a swing phase which is a state in which a foot is floating in the air and dividing the pieces of entire data for a unit step, thereby improving classification accuracy.

Furthermore, the present invention is directed to an apparatus and method for gait type classification using a pressure sensor of a smart insole, which are capable of removing variances of pieces of data according to a gait speed, performing data resizing for equal comparison between pieces of unit gait data to normalize pieces of divided data so as to equal lengths of the pieces of divided data, thereby accurately performing gait type classification of the pieces of gait data in which various variances are present.

Moreover, the present invention is directed to an apparatus and method for gait type classification using a pressure sensor of a smart insole, which are capable of effectively extracting discriminant characteristics from pieces of normalized data using a null-space linear discriminant analysis (Null-LDA) method when features are extracted for gait type classification.

It should be noted that objectives of the present invention are not limited to the above-described objectives, and other objectives of the present invention will be apparent to those skilled in the art from the following descriptions.

According to an aspect of the present invention, there is provided an apparatus for gait type classification including a gait data measuring part configured to measure pieces of gait data using a pressure sensor, a pre-processor configured to define a section of a unit step in all the pieces of gait data, divide the pieces of gait data for each unit step, and normalize the pieces of divided gait data to equalize lengths of the pieces of divided gait data, a feature extractor configured to extract features suitable for gait type classification from the pieces of pre-processed gait data, and a gait type classifier configured to receive the extracted features and determine and classify a final gait type.

A plurality of pressure sensors of the gait data measuring part may be provided at spaced positions in an insole of a shoe, and according to pressure strength, each of the plurality of pressure sensors may discriminate, measure, and store a state in which a foot is separated from the ground and a pressure is absent as “0,” a state in which a weak pressure is present as “1,” and a state in which a strong pressure is present as “2.”

The pre-processor may classify one gait cycle into a swing phase which is a state in which one foot floats in the air and a stance phase which is a state in which one foot is in contact with the ground, detect the swing phase, and construct a gait sample per unit step from all the pieces of measured gait data on the basis of the detected swing phase.

Since a foot is separated from the ground at a sampling point in the swing phase, criteria for detecting the swing phase and the stance phase, where all utilized values of the plurality of pressure sensors should be 0, may be defined as follows:

$$\{ \begin{matrix}SWING if p = 0 \\ STANCE if p > 0\end{matrix} \\ p = {\sum }_{i = 1}^{8} ( value of \text{i} \text{-} \text{th} pressure sensor ) ,$$

wherein p may denote the sum of all the values of the plurality of pressure sensors at a certain sampling point.

During the gait, the swing phase of the left foot may be detected, and on the basis of the detected swing phase of the left foot, a timing from a start point of the swing phase of the left foot to an end point of the stance phase may be defined as one piece of step data.

The pre-processor may remove sections each having a length of 0.5 seconds or less among sections for one step by regarding such sections as being generated by a false positive (FP) swing phase.

The pre-processor may measure a shortest time among the pieces of noise-removed unit step data, resize each of the pieces of unit step data to have a length corresponding to a measured unit time, and normalize the sections for one step to have the same size.

The feature extractor may extract discriminant features from the pieces of normalized unit gait data using the Null-LDA method when the discriminant features are extracted for gait type classification.

The Null-LDA method may be a method in which a within-class scatter matrix S_{W} and a between-class scatter matrix S_{B} of N pieces of learning data xₖ constituted with C classes may respectively be defined as

$${S}_{W} = {{\sum }^{C}}_{i = 1} {\sum }_{{x}_{k} ∈ {c}_{i}} ( {x}_{k} - {μ}_{i} ) {( {x}_{k} - {μ}_{i} )}^{T} and$$

$${S}_{B} = \frac{1}{N} {\sum }_{i = 1}^{C} {N}_{i} ( {μ}_{i} - μ ) {( {μ}_{i} - μ )}^{T} ,$$

and an objective function may be set as

$${W}_{LDA} = {argmax}_{W} \frac{\vert {W}^{T} {S}_{B} W \vert}{\vert {W}^{T} {S}_{W} W \vert}$$

to maximize a ratio of a variance of the within-class scatter matrix S_{W} to a variance between averages of the between-class scatter matrix S_{B}.

W_{LDA} satisfying the objective function may be calculated by eigenvalue analysis of S_{W}⁻¹S_{B}, and a feature yₖ of a sample xₖ may be calculated as W_{LDA}^{T}xₖ using W_{LDA}.

When a small sample size (SSS) problem, in which the number of samples is smaller than the number of dimensions of pieces of data, S_{W}⁻¹ is not present and thus a solution is not calculated, occurs, a kernel principal component analysis (PCA)+LDA method, in which the PCA is applied to decrease the number of dimensions of pieces of data to less than that of the scatter matrix S_{W} and then an LDA method is applied, may be used, and the Null-LDA method of projecting a within-class data into a null space and then searching a subspace where a scatter matrix is maximized is applied.

When a total scatter matrix S_{T} is defined as

$${S}_{T} = {\sum }_{i = 1}^{C} {\sum }_{{x}_{k} ∈ {c}_{i}} ( {x}_{k} - μ ) {( {x}_{k} - μ )}^{T} ,$$

a projective matrix of the PCA+LDA method may be W_{PCA+LDA}=W_{LDA}^{T}W_{PCA}^{T}, wherein W_{PCA}=argmax_{W}|W^{T}S_{T}W| and

$${W}_{LDA} = arg {max }_{W} \frac{\vert {W}^{T} {W}_{PCA}^{T} {S}_{B} {W}_{PCA} W \vert}{\vert {W}^{T} {W}_{PCA}^{T} {S}_{W} {W}_{PCA} W \vert} ,$$

and the Null-LDA method may calculate a projective matrix W_{NLDA} satisfying the objective function of W_{NLDA}=argmax_{|W}_{^{T}}_{S}_{_{W}}_{W|=0}|W^{T}S_{B}W| in a space in which |W^{T}S_{W}W|=0 and |W^{T}S_{B}S|≠0 using a null space of the within-class scatter matrix S_{W}.

The gait data measuring part may further include an accelerometer sensor provided in an insole of a shoe, and the feature extractor may extract the features suitable for user identification from the pieces of pre-processed data, wherein the apparatus further includes a user identifier configured to receive the features suitable for the user identification extracted by the feature extractor and perform the user identification.

The Null-LDA method may define a between-class scatter matrix S_{B} and a within-class scatter matrix S_{W} for N samples constituted with C classes as

$${S}_{B} = \frac{1}{N} {\sum }_{i = 1}^{C} {N}_{i} ( {μ}_{i} - μ ) {( {μ}_{i} - μ )}^{T} and$$

$${S}_{W} = {{\sum }^{C}}_{i = 1} {\sum }_{{x}_{m} ∈ {c}_{i}} ( {x}_{m} - {μ}_{i} ) {( {x}_{m} - {μ}_{i} )}^{T} ,$$

respectively, wherein xₘϵR^{n×1} may be an m^{th} sample belonging to a class Cᵢ, and μ and μᵢ may refer to an average of all samples and an average of samples belonging to class Cᵢ, respectively.

The Null-LDA method may project the samples into the null space of the within-class scatter matrix S_{W} so as to maximize a discriminant power between classes and calculate projective vectors satisfying the objective function using W_{NLDA}=argmax_{|W}_{^{T}}_{S}_{_{W}}_{W|=0}|W^{T}S_{B}W| so as to maximize a variance between averages of the classes, and W_{NLDA} may be a projective matrix constituted with wₜ, t=1, . . . n′ of n′ projective vectors.

A feature vector y for a sample x may be y=W^{T}ₓ (ϵR^{n×1}).

According to another aspect of the present invention, there is provided a method for gait type classification using a pressure sensor of a smart insole includes measuring pieces of gait data using a plurality of pressure sensors provided at spaced positions in an insole of a shoe, removing sections each having a length less than a preset time among sections for one step by regarding the sections each having a length less than the preset time as being generated by a false positive (FP) swing phase, pre-processing the pieces of gait data by measuring a shortest time among the pieces of noise-removed unit step data, resizing each of the pieces of unit step data to a length corresponding to a measured unit time, and normalizing sections for one step to have the same size, and extracting features suitable for gait type classification from the pieces of pre-processed data, receiving the extracted features as an input, and determining and classifying a final gait type.

The measuring of the pieces of gait data may include classifying, measuring, and storing, according to pressure strength, a state in which a foot is separated from the ground and a pressure is absent as “0,” a state in which a weak pressure is present as “1,” and a state in which a strong pressure is present as “2.”

The pre-processing of the pieces of gait data may include classifying one gait cycle into a swing phase in a state in which one foot is floating in the air and a stance phase in a state in which one foot is in contact with the ground and detecting the swing phase and constituting a gait sample per unit step from all the pieces of measured gait step data on the basis of the detected swing phase.

Since a foot is separated from the ground at a sampling point in the swing phase, criteria for detecting the swing phase and the stance phase, where all utilized values of the plurality of pressure sensors should be 0, may be defined as

$$\{ \begin{matrix}SWING if p = 0 \\ STANCE if p > 0\end{matrix} \\ p = {\sum }_{i = 1}^{8} ( value of \text{i} \text{-} \text{th} pressure sensor ) ,$$

wherein p may denote the sum of all the values of the plurality of pressure sensors a certain sampling point.

During the gait, the swing phase of the left foot may be detected, and on the basis of the detected swing phase of the left foot, a timing from a start point of the swing phase of the left foot to an end point of the stance phase may be defined as one piece of step data.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of an apparatus for gait type classification using a pressure sensor according to a related art;

FIG. 2 is a block diagram of an apparatus for gait type classification using an accelerometer sensor and a gyroscope sensor according to the related art;

FIG. 3 is a diagram illustrating a gait cycle;

FIG. 4 is a block diagram of an apparatus for gait type classification using a pressure sensor of a smart insole according to the present invention;

FIG. 5 is a flowchart illustrating a method for gait type classification using a pressure sensor of a smart insole according to the present invention;

FIGS. 6A to 6H are graphs showing measured pressure values of respective sensors of both insoles;

FIG. 7 is a diagram illustrating an example of normalization of pieces of step data;

FIG. 8 is a graph showing a change in classification accuracy according to the number of steps per sample;

FIG. 9 is a diagram illustrating an example of pieces of gait data measured using a gait data measurement sensor;

FIG. 10 is a flowchart illustrating a method for user identification using an accelerometer sensor; and

FIG. 11 is a graph showing a recognition rate according to the number of dimensions of a null-space linear discriminant analysis (Null-LDA) feature space for 568 gait samples formed by dividing a total of 2295 steps into four consecutive steps.

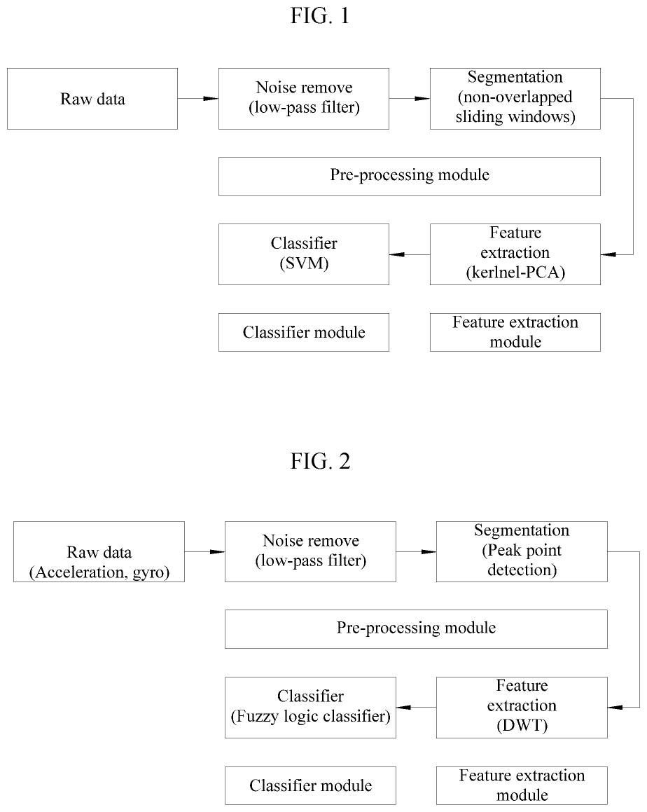

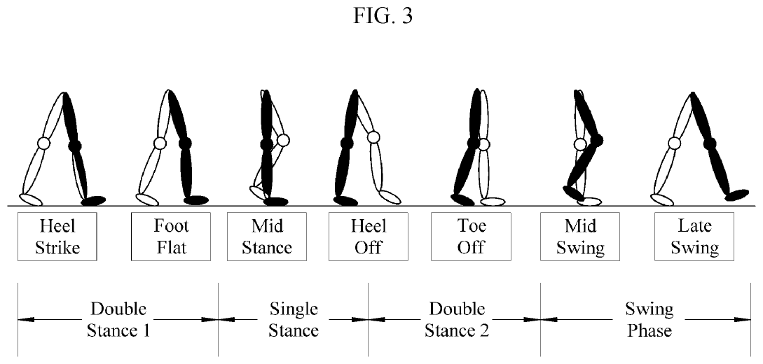

### DETAILED DESCRIPTION

Hereinafter, exemplary embodiments of an apparatus and a method for gait type classification using a pressure sensor of a smart insole according to the present invention will be described in detail below.

The features and advantages of the apparatus and a method for gait type classification using a pressure sensor of a smart insole according to the present invention will be apparent from the following detailed description of each embodiment.

FIG. 3 is a diagram illustrating a gait cycle.

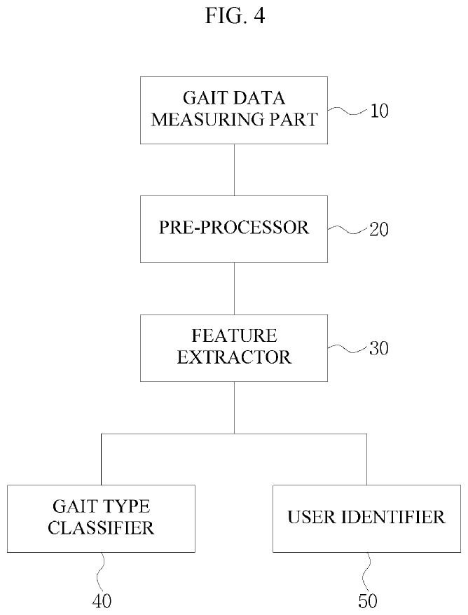

Further, FIG. 4 is a block diagram of an apparatus for gait type classification using a pressure sensor of a smart insole according to the present invention.

The apparatus and a method for gait type classification using a pressure sensor of a smart insole according to the present invention is capable of classifying types of pieces of gait data having various variances using only a pressure sensor.

To this end, the present invention may include a preprocessing configuration for removing noise from pieces of measured data and alleviating various variances such as a change in gait speed and in gait environment including a terrain even though gait types are the same.

The preprocessing configuration may include a configuration for removing sections each having a length of 0.5 seconds or less among sections for one step by regarding the sections each having the length of 0.5 seconds or less as being generated by a false positive (FP) swing phase, for measuring a shortest time among pieces of noise-removed unit step data, and for resizing each of the pieces of unit step data to a length corresponding to a measured unit time to normalize the sections for one step to have the same size.

A pressure sensor for acquiring pieces of gait data in the present invention may employ a FootLogger smart insole which is a commercially available smart insole developed by 3L-Labs Co., Ltd. (Seoul Korea), but the pressure sensor is not limited thereto.

The FootLogger smart insole has eight pressure sensors in one insole, and each of the eight pressure sensors stores a value of 0, 1, or 2 according to pressure strength.

As shown in FIG. 3, the gait cycle may be divided into seven stages, i.e., a heel strike, a foot flat, a mid stance, a heel off, a toe off, a mid swing, and a late swing.

The heel strike stage is the beginning of the gait cycle and is in a state in which a heel is in contact with the ground, the foot flat stage is in a state in which an entire sole is in contact with the ground, the mid stance stage is in a state until the center of a body moves to a front foot, and the heel off stage and the toe off stage are in a state in which the heel and toes are separated from the ground, the mid swing stage and the late swing stage are in a state in which the foot is floating in the air, and one gait cycle ends in the late swing stage.

Alternatively, one gait cycle may be classified into a swing phase in a state in which one foot is floating in the air, and a stance phase in a state in which one foot is in contact with the ground.

Conventionally used gait classifiers use a sliding window method for rapid gait type classification.

However, even though gait types are the same, gait speeds may be slightly different according to a person or a situation, and variances of the gait types cause difficulty in determining the gait types from pieces of collected data.

The present invention detects the swing phase and constitutes a gait sample for each unit step from pieces of an entirety of measured step data on the basis of the detection.

First, the swing phase is detected from the pieces of entire step data, and a detection criterion is as follows.

Table 1 shows some pieces of data measured using pressure sensors of a FootLogger insole.

The FootLogger smart insole has eight pressure sensors, and each of the eight pressure sensors measures pieces of data at a sampling rate of 100 Hz.

Each of the eight pressure sensors stores a value of 0, 1, or 2 according to pressure strength.

0 denotes a state in which a pressure is absent (a foot is separated from the ground), 1 denotes a state in which a weak pressure present, and 2 denotes a state in which a strong pressure is present.

In Table 1, a row indicates an index of each of the eight pressure sensor, and a column indicates a sampling point at an interval of 0.01 seconds.

TABLE 1

| Time | Sensor1 | Sensor2 | Sensor3 | Sensor4 | Sensor5 | Sensor6 | Sensor7 | Sensor8 |
|---|---|---|---|---|---|---|---|---|
| 2017-07-31 | 0 | 0 | 0 | 2 | 0 | 0 | 0 | 2 |
| 17:39:28.748 | | | | | | | | |
| 2017-07-31 | 0 | 0 | 0 | 2 | 0 | 0 | 0 | 2 |
| 17:39:28.758 | | | | | | | | |
| 2017-07-31 | 0 | 0 | 0 | 2 | 0 | 0 | 0 | 2 |
| 17:39:28.768 | | | | | | | | |
| 2017-07-31 | 0 | 0 | 0 | 2 | 0 | 0 | 0 | 2 |
| 17:39:28.778 | | | | | | | | |
| 2017-07- | 0 | 0 | 0 | 2 | 0 | 0 | 0 | 2 |
| 3117:39:28.788 | | | | | | | | |
| 2017-07- | 0 | 0 | 0 | 2 | 0 | 0 | 0 | 2 |
| 3117:39:28.798 | | | | | | | | |
| 2017-07-31 | 0 | 0 | 0 | 2 | 0 | 0 | 0 | 2 |
| 17:39:28.808 | | | | | | | | |
| 2017-07-31 | 0 | 0 | 0 | 2 | 0 | 0 | 0 | 2 |
| 17:39:28.818 | | | | | | | | |
| 2017-07-31 | 0 | 0 | 0 | 2 | 0 | 0 | 1 | 2 |
| 17:39:28.828 | | | | | | | | |
| 2017-07-31 | 0 | 0 | 0 | 2 | 0 | 0 | 1 | 2 |
| 17:39:28.838 | | | | | | | | |
| 2017-07-31 | 0 | 0 | 0 | 2 | 0 | 0 | 1 | 2 |
| 17:39:28.848 | | | | | | | | |
| 02017-07-31 | 0 | 0 | 0 | 2 | 0 | 0 | 1 | 2 |
| 17:39:28.858 | | | | | | | | |
| 2017-07-31 | 0 | 0 | 0 | 2 | 0 | 0 | 1 | 2 |
| 17:39:28.868 | | | | | | | | |
| 2017-07-31 | 0 | 0 | 0 | 2 | 0 | 0 | 1 | 2 |
| 17:39:28.878 | | | | | | | | |
| 2017-07-31 | 1 | 1 | 0 | 2 | 1 | 0 | 0 | 2 |
| 17:39:28.888 | | | | | | | | |
| 2017-07-31 | 1 | 1 | 0 | 2 | 1 | 0 | 0 | 2 |
| 17:39:28.898 | | | | | | | | |
| 2017-07-31 | 1 | 1 | 0 | 2 | 1 | 0 | 0 | 2 |
| 17:39:28.908 | | | | | | | | |
| 2017-07-31 | 2 | 2 | 0 | 2 | 1 | 0 | 0 | 2 |
| 17:39:28.918 | | | | | | | | |
| 2017-07-31 | 2 | 2 | 0 | 1 | 1 | 0 | 0 | 1 |
| 17:39:28.928 | | | | | | | | |
| 2017-07-31 | 2 | 2 | 0 | 1 | 1 | 0 | 0 | 1 |
| 17:39:28.938 | | | | | | | | |
| 2017-07-31 | 2 | 2 | 0 | 1 | 1 | 0 | 0 | 1 |
| 17:39:28.948 | | | | | | | | |
| 2017-07-31 | 2 | 2 | 0 | 1 | 1 | 0 | 0 | 1 |
| 17:39:28.958 | | | | | | | | |
| 2017-07-31 | 2 | 2 | 0 | 1 | 1 | 0 | 0 | 1 |
| 17:39:28.968 | | | | | | | | |
| 2017-07-31 | 2 | 2 | 0 | 1 | 1 | 0 | 0 | 1 |
| 17:39:28.978 | | | | | | | | |
| 2017-07-31 | 2 | 2 | 0 | 1 | 1 | 0 | 0 | 0 |
| 17:39:28.988 | | | | | | | | |
| 2017-07-31 | 2 | 2 | 0 | 1 | 1 | 0 | 0 | 0 |
| 17:39:28.998 | | | | | | | | |
| 2017-07-31 | 2 | 2 | 0 | 0 | 1 | 0 | 0 | 0 |
| 17:39:29.008 | | | | | | | | |
| 2017-07-31 | 2 | 2 | 0 | 0 | 1 | 0 | 0 | 0 |
| 17:39:98.018 | | | | | | | | |
| 2017-07-31 | 2 | 2 | 0 | 0 | 1 | 0 | 0 | 0 |
| 17:39:28.018 | | | | | | | | |

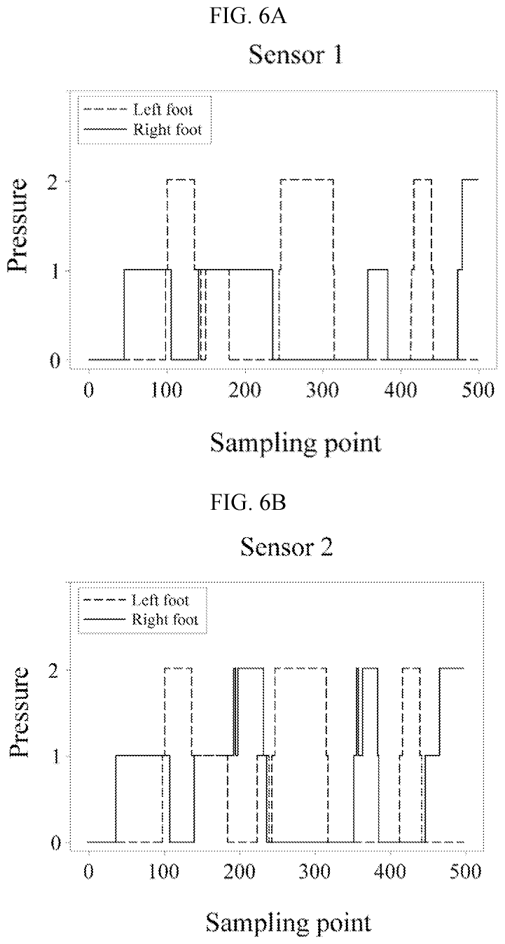

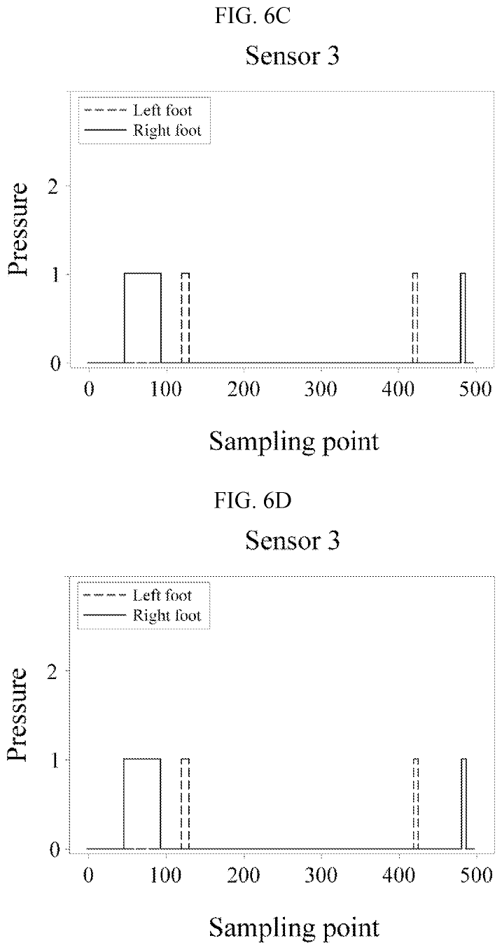

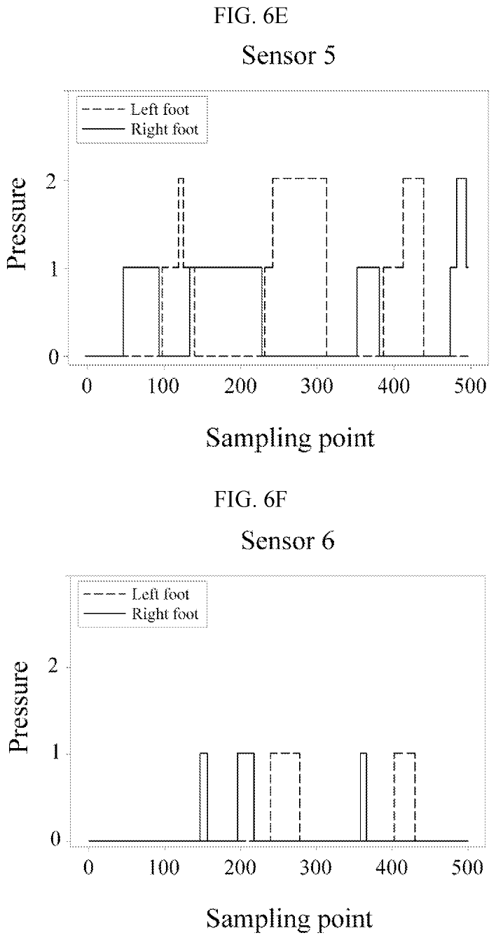

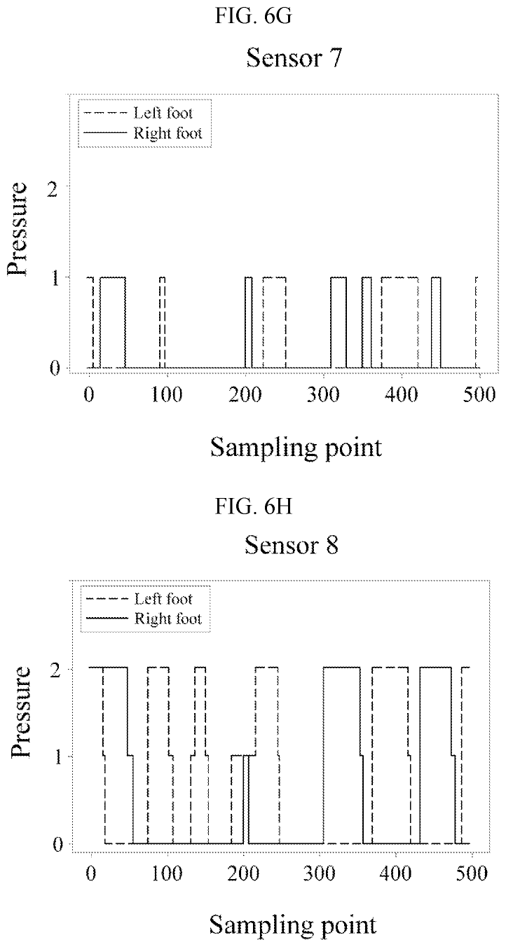

FIGS. 6A to 6H are graphs showing measured pressure values of respective sensors of both insoles.

Since the foot is separated from the ground at a sampling point in the swing phase, criteria for detecting the swing phase and the stance phase, where all utilized values of the eight pressure sensors should be 0, may be determined as follows.

$$\{ \begin{matrix}SWING if p = 0 \\ STANCE if p > 0\end{matrix} \\ p = {\sum }_{i = 1}^{8} ( value of \text{i} \text{-} \text{th} pressure sensor ) \qquad [ Equation 1 ]$$

Here, p denotes the sum of all values of the eight pressure sensors at a certain sampling point.

FIGS. 6A to 6H are graphs showing measured pressure values of the eight pressure sensors of both insoles, and during a gait, it can be seen that a swing phase of a left foot (at a state in which the left foot is separated from the ground, i.e., a section in which a value of each of the eight pressure sensors is 0) and a swing phase of a right foot alternate.

In the present invention, during the gait, the swing phase of the left foot is detected, and on the basis of the detected swing phase of the left foot, a timing from a start point of the swing phase of the left foot to an end point of the stance phase is defined as one piece of step data.

As shown in FIG. 4, the apparatus for gait type classification using a pressure sensor of a smart insole according to the present invention includes a gait data measuring part 10 for measuring pieces of gait data using only a pressure sensor, a pre-processor 20 for removing sections each having a length of 0.5 seconds or less among sections for one step by regarding the sections each having a length of 0.5 seconds or less as being generated by an FP swing phase, for measuring a shortest time among pieces of noise-removed unit step data, and for resizing each of the pieces of unit step data to a length corresponding to a measured unit time to normalize the sections for one step to have the same size, a feature extractor 30 for extracting features suitable for the gait type classification from the pieces of preprocessed data, and a gait type classifier 40 for receiving the extracted features as an input and determining and classifying a final gait type.

Here, the removing of the noise in the pre-processor 20 is performed as follows.

The pieces of gait data measured by the gait data measuring part 10 may include noise due to factors such as a measurement environment, a potential difference in a module, and heat of the eight pressure sensors.

When even one among the eight pressure sensors in the swing phase exhibits a measured value which is not zero due to such noise, this causes the FP swing phase.

Further, the sections for one step are constituted by the swing phase such that overall performance of the gait type classifier 40 may be degraded.

Since swing phases corresponding to an FP are each shorter in length than normal swing phases, the present invention regards and removes the section each having a length of 0.5 seconds or less as being generated by the FP swing phase.

Table 2 shows the results of comparing the number of steps measured with the normal swing phases by removing sections for one step which are generated by the FP according to the present invention.

TABLE 2

| | WK | RA | RD | SA | SD | RUN | FWK |
|---|---|---|---|---|---|---|---|
| Steps measured | 176 | 109 | 114 | 55 | 64 | 252 | 186 |
| Actual steps | 176 | 114 | 114 | 55 | 65 | 244 | 186 |

Here, the gait types includes walking (WK), fast run (FWK), run (RUN), uphill (RA), downhill (RD), climbing stairs (SA), and step down (SD).

Further, gait data normalization in the pre-processor 20 is performed as follows.

Three factors, including a physical factor, a geographical factor, and a speed factor, affect the gait type. The physical factor includes the age and health status of a pedestrian, the geographical factor includes the slope and shape of a terrain, and the speed factor includes a situation for when the pedestrian is walking or a difference in individual speed.

Among these three factors, the speed factor affects a size of the pieces of gait data to cause difficulty in performing various discriminant analysis using divided samples of the pieces of unit step data.

In the present invention, after a shortest time t* of the pieces of noise-removed unit step data is measured, all the pieces of unit step data are resized to a length corresponding to the measured unit time such that the sections for one step are normalized to have the same size.

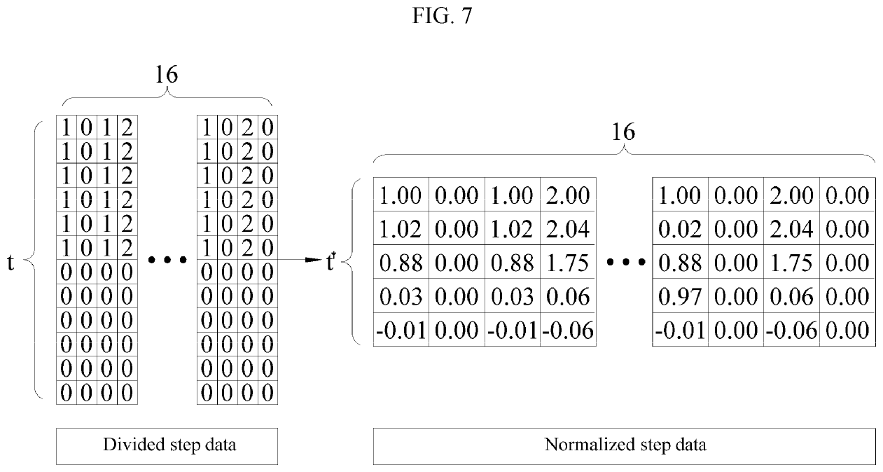

FIG. 7 is a diagram illustrating an example of normalization of the pieces of gait data.

$$\text{normalized point =} \frac{non normalized point}{length of non normalized signal} length of normalized signal \qquad [ Equation 2 ]$$

An LDA method performed in the feature extractor 30 will be described below.

The LDA method is a widely used method of extracting features for a multi-class classification problems, and a within-class scatter matrix S_{W} and a between-class scatter matrix S_{B} of N pieces of learning data xₖ constituted with C classes are respectively defined as Equations 4 and 5, and an objective function is set as Equation 6 so as to maximize a ratio of a within-class variance to a variance between between-class averages.

$$\begin{matrix}{μ}_{i} = \frac{1}{\vert {C}_{i} \vert} {\sum }_{{x}_{k} ∈ {C}_{i}} {x}_{k} & [ Equation 3 ] \\ {S}_{W} = {\sum }_{i = 1}^{C} {\sum }_{{x}_{k} ∈ {c}_{i}} ( {x}_{k} - {μ}_{i} ) {( {x}_{k} - {μ}_{i} )}^{T} & [ Equation 4 ] \\ {S}_{B} = \frac{1}{N} {\sum }_{i = 1}^{C} {N}_{i} ( {μ}_{i} - μ ) {( {μ}_{i} - μ )}^{T} & [ Equation 5 ] \\ {W}_{LDA} = arg {max}_{W} \frac{\vert {W}^{T} {S}_{B} W \vert}{\vert {W}^{T} {S}_{W} W \vert} & [ Equation 6 ]\end{matrix}$$

W_{LDA} satisfying the objective function of Equation 6 may be calculated by eigenvalue analysis of S_{W}⁻¹S_{B}, and a feature yₖ of the N pieces of learning data may be calculated as W_{LDA}^{T}xₖ using with respect to the sample xₖ.

Meanwhile, when the number of the N pieces of learning data is less than a dimension for the N pieces of learning data, S_{W}⁻¹ is not present and thus a solution of Equation 6 cannot be calculated such that a small sample size (SSS) problem occurs.

In order to resolve the SSS problem, a principal component analysis (PCA)+LDA method may be used to apply the PCA, decrease the dimension for the N pieces of learning data to less than a dimension of the within-class scatter matrix S_{W}, and apply the LDA, or the Null-LDA method may be used to project pieces of within-class data into a null space and search a subspace in which the within-class scatter matrix S_{W} is maximized.

A total scatter matrix ST is calculated as Equation 7.

$$\begin{matrix}{S}_{T} = {\sum }_{i = 1}^{C} {\sum }_{{x}_{k} ∈ {c}_{i}} ( {x}_{k} - μ ) {( {x}_{k} - μ )}^{T} & [ Equation 7 ] \\ {W}_{PCA + LDA} = {W}_{LDA}^{T} {W}_{PCA}^{T} \\ Here , {W}_{PCA} = {argmax}_{W} \vert {W}^{T} {S}_{T} W \vert and , {W}_{LDA} = {argmax}_{W} \frac{\vert {W}^{T} {W}_{PCA}^{T} {S}_{B} {W}_{PCA} W \vert}{\vert {W}^{T} {W}_{PCA}^{T} {S}_{W} {W}_{PCA} W \vert} . & [ Equation 8 ]\end{matrix}$$

The null-LDA method calculates a projective matrix W_{NLDA} which satisfies the following objective function in a space of |W^{T}S_{W}W|=0 and |W^{T}S_{B}W|≠0 using the null space of the within-class scatter matrix S_{W}.

W_{NLDA}=argmax_{|W}_{^{T}}_{S}_{_{W}}_{W|=0}|W^{T}S_{B}W|[Equation 9]

As described above, the Null-LDA method exhibits high performance in classification of pieces of high-dimensional data in which the null space of the within-class scatter matrix S_{W} occurs sufficiently, and in the present invention, the Null-LDA method may be used as a linear discriminant analysis method for gait type classification.

An operation of the apparatus for gait type classification using a pressure sensor of a smart insole according to the present invention having the above-described configuration will be described in detail below.

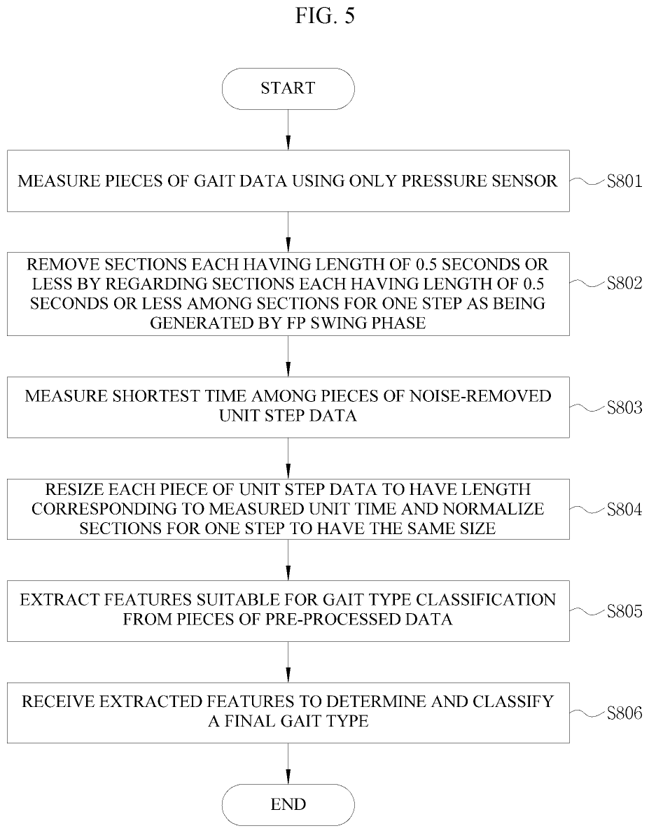

FIG. 5 is a flowchart illustrating a method for gait type classification using a pressure sensor of a smart insole according to the present invention.

The method for gait type classification using a pressure sensor of a smart insole according to the present invention includes a pre-processing operation of removing noise from pieces of data, dividing the pieces of data into a unit step, and forming a unit step sample, a feature extraction operation of extracting features suitable for classification from the unit step sample, and a classification operation using the extracted features.

In the pre-processing, a section corresponding to one step is first defined in entire pieces of data by detecting a swing phase which is a state in which a foot floats in the air, and the pieces of data for each unit step are divided.

Then, variances of the pieces of data due to a gait speed are removed, and data resizing is performed for equal comparison between the pieces of unit gait data to normalize the pieces of unit gait data, thereby equalizing lengths of the pieces of divided data.

In the feature extraction operation, discriminant features are extracted from the pieces of normalized data using a Null-LDA method.

Specifically, the pieces of gait data are measured using only the pressure sensor (S801).

Then, sections each having a length of 0.5 seconds or less among sections for one step are regarded as being generated by an FP swing phase and are removed (S802).

A shortest time among the pieces of noise-removed unit step data is measured (S803), and each of the pieces of unit step data is resized to a length corresponding to a measured unit time such that the sections for one step are normalized to have the same size (S804).

Then, a feature suitable for the gait type classification is extracted from the pieces of pre-processed data (S805), and the extracted feature is received to determine and classify a final gait type (S806).

In order to evaluate performance of the apparatus and the method for gait type classification using a pressure sensor of a smart insole according to the present invention, the following tests were carried out.

In one embodiment of the present invention, the FootLogger smart insole, which is a commercially available smart insole manufactured by 3L-Labs Co., Ltd. (Seoul, Korea), was used for gait data collection.

The FootLogger smart insole has eight pressure sensors, a 3-axis accelerometer sensor, and a 3-axis gyroscope sensor and measures a gait type by being worn on shoes of both feet.

Pieces of sensed data of the FootLogger smart insole are stored in a smartphone via Bluetooth.

In order to acquire a data set in which a geographical variance and a speed variance are large, pieces of data were collected in cases of walking or running on flat ground, stairs, and hill terrain.

There were seven gait types of a WK on the flat ground, race walking, a RUN, an RA, an RD, an SA, and an SD, and when the pieces of gait data were collected, a gait was performed at a natural speed.

Each of the pieces of gait data was pre-processed to be stored in an 816-dimensional vector form.

TABLE 3

| terrain | gait type | number of steps | measuring time | |
|---|---|---|---|---|
| flat | walking (WK) | 2193 | 3 | mins |
| hill | uphill (RA) | 1493 | 2 | mins |
| hill | downhill (RD) | 1483 | 2 | mins |
| stairs | climbing stairs (SA) | 760 | 1 | min |
| stairs | step down (SD) | 838 | 1 | min |
| flat | run (RUN) | 3154 | 3 | mins |
| flat | fast run (FWK) | 2530 | 3 | mins |

Table 3 shows information on measured data samples.

In order to evaluate performance of the method for gait type classification according to the present invention, the pieces of gait data acquired using the FootLogger smart insole were pre-processed and a classification test was performed.

The Null-LDA method was used as a feature extraction method based on linear discriminant analysis.

After randomly mixing all data samples, a classification rate was measured using half of the data samples as learning data and the other half as test data, and this test was repeated 20 times to calculate an average classification rate.

Prior to extracting Null-LDA features, all the data samples were normalized to have a zero-mean and a unit standard deviation with an average and a standard deviation of the learning data, and a nearest neighborhood (NN) rule classifier was used as a classifier using the extracted Null-LDA features.

The pieces of pre-processed gait data include a total of 760 to 3154 steps according to the gait type.

First, in order to find out how many steps are needed to extract a meaningful gait type in a Null-LDA feature space, a classification test was carried out from a case in which one step is constituted with one gait sample to a case in which six steps are constituted with one gait sample.

When one sample is constituted with one step, the gait data sample is stored as an 816-dimensional vector, and when one sample is constituted with six steps, the gait data sample is stored as a 4896-dimensional vector.

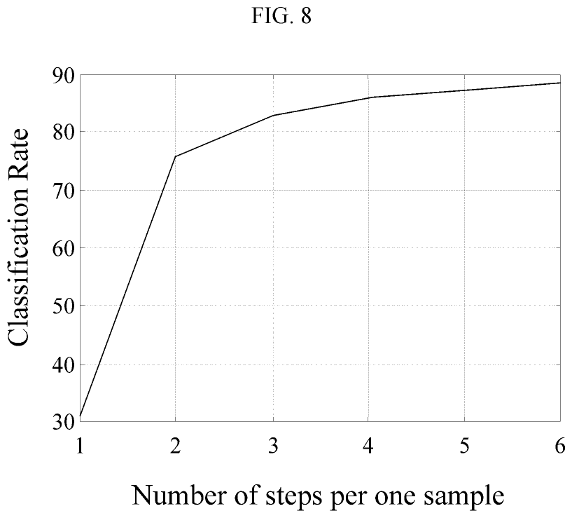

FIG. 8 is a graph showing a change in classification accuracy according to the number of steps per sample.

When the features were extracted at two steps, the classification performance was significantly improved more than when the features were extracted at one step so that it was determined that a meaningful gait type was found when two or more steps are performed.

Further, as one sample includes a larger number of steps, the classification performance is gradually improved, so it is determined that as the dimension of the sample becomes larger, the null space of the within-class scatter matrix S_{W} is sufficiently secured in a process of finding a feature space of the Null-LDA.

Unlike the conventional method using a real valued pressure measurement value, the present invention used only pressure measurement values of at 0, 1, and 2 stages to exhibit high classification performance of 88% with respect to three gait types: the WK, the SA, and the SD.

Meanwhile, the gait data measuring part 10 of the apparatus for gait type classification of the present invention further includes an accelerometer sensor provided at an insole of the shoe, and the feature extractor 30 extracts features suitable for user identification from the pieces of pre-processed data. Also, a user identifier 50 may be further included to receive the features suitable for user identification from the user extractor 30 and perform the user identification.

Thus, the apparatus for gait type classification of the present invention acquires the pieces of gait data using the accelerometer sensor of the smart insole and recognizes a user based on discriminant analysis for the pieces of gait data. Since a wearable sensor is used in the present invention, it is possible to efficiently identify the user because there is hardly any restriction on a use environment, such as the user should be in the crowd or a specific place.

The FootLogger smart insole has eight pressure sensors, a 3-axis accelerometer sensor, and a 3-axis gyroscope sensor, and each of the sensors installed in both shoes measure pieces of data at a sampling rate of 100 Hz.

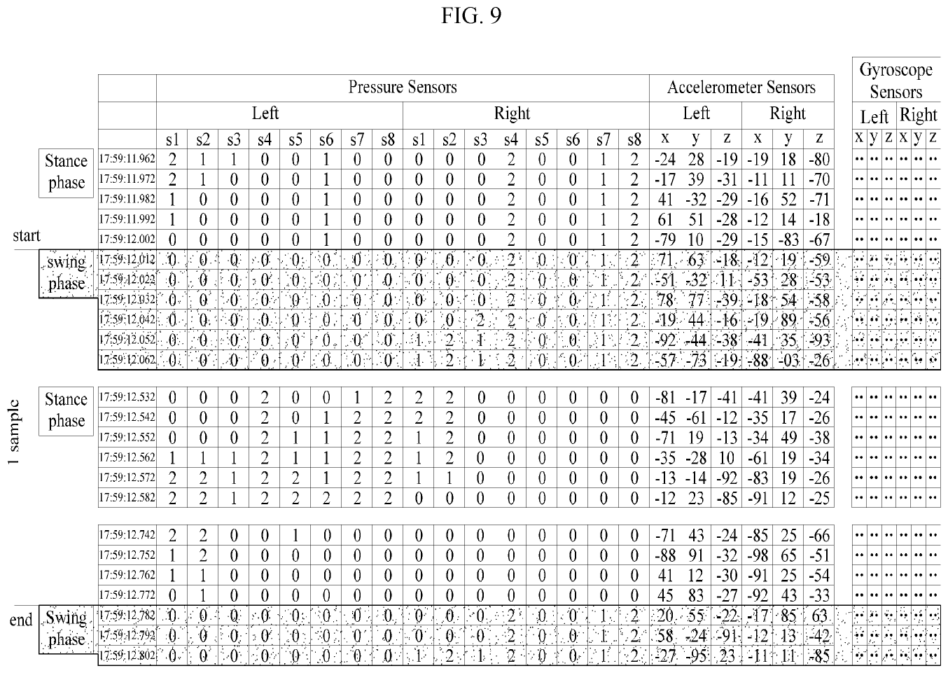

FIG. 9 is a diagram illustrating an example of pieces of gait data measured using the gait data measurement part 10. The example of the pieces of gait data measured using the FootLogger smart insole is shown, a gait cycle may be confirmed through the swing phase which is alternatively shown at a left foot and a right foot, and in the present invention, one step is defined from a start point of the swing phase to an end point of a stance phase about the left foot during the user identification.

Meanwhile, even for the same person, a gait speed may be varied during when pieces of data are measured or during every attempt to measure the pieces of data, and such a change in gait speed hinders extraction of an individual gait characteristic for user identification. Thus, in order to extract features which are less sensitive to situations, all unit steps from the swing phase to the stance phase are resized on the basis of a time t of a shortest unit step (which is set as t=63 in one embodiment of the present invention), thereby being normalized to an array of 63×6 so as to equal lengths for the unit steps. A measured acceleration value of the normalized unit step is stored as a vector x of 378×1 using a lexicographic ordering operator.

In this case, it is preferable to exclude pieces of data for the unit steps of which a value of each of the pressure sensors is not 0 in the swing phase due to malfunction of the measurement sensors during the data pre-processing.

The feature extractor 30 may use a Null-LDA method to extract features suitable for user identification from the pieces of gait data pre-processed in the pre-processor 20.

First, for N samples constituted with C classes, a between-class scatter matrix SB and a within-class scatter matrix SW are defined as follows.

$$\begin{matrix}{S}_{B} = \frac{1}{N} {\sum }_{i = 1}^{C} {N}_{i} ( {μ}_{i} - μ ) {( {μ}_{i} - μ )}^{T} & [ Equation 10 ] \\ {S}_{W} = {\sum }_{i = 1}^{C} {\sum }_{{x}_{m} ∈ {c}_{i}} ( {x}_{m} - {μ}_{i} ) {( {x}_{m} - {μ}_{i} )}^{T} & [ Equation 11 ]\end{matrix}$$

Here, xₙϵR^{n×1} is an m^{th} sample belonging to a class Cᵢ, and μ and μᵢ mean an average of entire samples and an average of the samples belonging to class Cᵢ, respectively.

In order to maximize discrimination power between classes, the Null-LDA method projects the samples into a null space of the within-class scatter matrix S_{W} and then calculates projective vectors satisfying the following objective function so as to maximize a variance between the averages of the classes.

W_{NLDA}=argmax_{|W}_{^{T}}_{S}_{_{W}}_{W|=0}|W^{T}S_{B}W| [Equation 12]

W_{NLDA} is a projective matrix constituted with n′ projective vectors wₜ, t=1, . . . n, and a feature vector y for a sample x may be calculated as follows.

y=W^{T}x(ϵR^{n×1}) [Equation 13]

An operation of the user identification using the apparatus for gait type classification, which has the above-described configuration, according to the present invention will be described below.

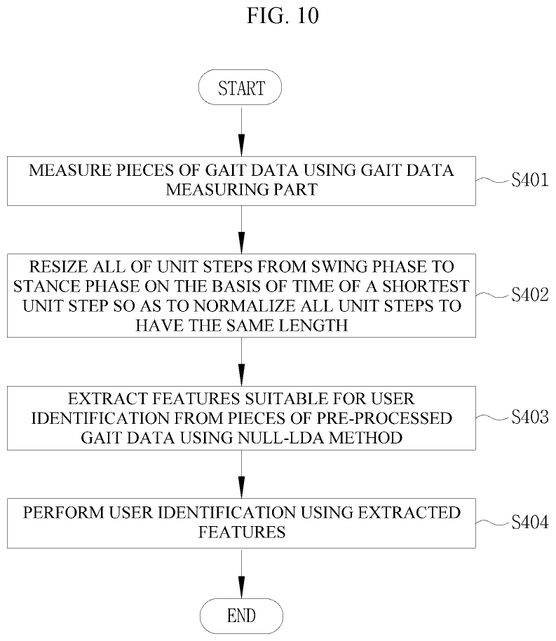

FIG. 10 is a flowchart illustrating a method for user identification using the apparatus for gait type classification according to the present invention.

In the method for user identification according to the present invention, pieces of gait data are first measured using the gait data measuring part 10 (S401).

Then, all unit steps from the swing phase to the stance phase are resized on the basis of a time of a shortest unit step, and a pre-processing for normalization is performed on all unit steps to equal lengths of the unit steps to have the same length (S402). Thereafter, features suitable for the user identification are extracted from the pieces of pre-processed gait data using the Null-LDA method (S403), and the user identification is performed using the extracted features (S404).

Performance of the above-described user identification of the present invention will be described below.

A recognition test was performed by pre-processing the measured acceleration values of the FootLogger smart insole and with the features extracted using the Null-LDA method.

A one-NN method using a “Euclidean distance” was used as a classifier.

A learning data set was constituted by randomly selecting three samples from data samples of each person, and the remaining data samples were used a test data set.

An average of recognition rates obtained by repeating the recognition test 25 times was shown as a result of the recognition test.

TABLE 4

| | No. of steps in a single sample | | | | |
|---|---|---|---|---|---|
| | 1 | 2 | 3 | 4 | 5 |
| Recog. rate (%) | 86.9 | 89.3 | 91.2 | 93.5 | 93.9 |

First, in order to determine how many steps are required to extract gait features for the user identification, recognition rates from a gait sample constituted with the measured acceleration value for one step to a gait sample including measured values of five steps were measured.

The recognition rates of Table 4 were measured by randomly selecting 400 samples among all samples.

In Table 4, it can be seen that the recognition rate was 87% with only one step, and as the number of steps included in the gait sample increased, the recognition rate was increased and then width an increment slows down at five steps. This means that features of an individual gait type are extracted more effectively when walking over four steps.

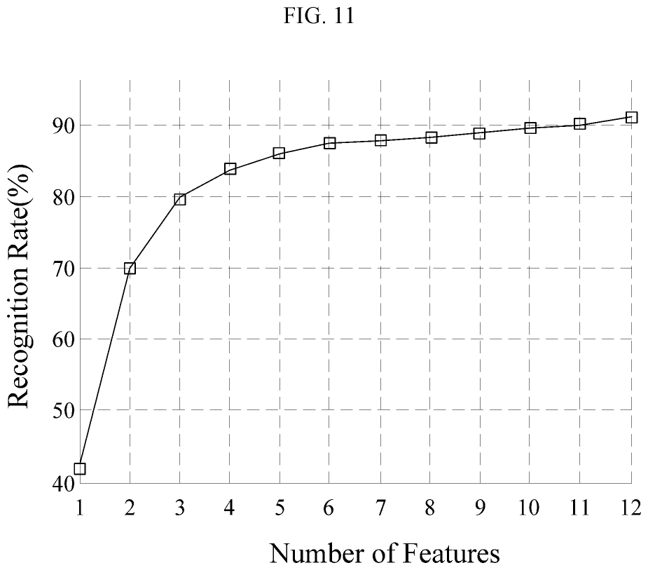

FIG. 11 is a graph showing a recognition rate according to the number of dimensions of a Null-LDA feature space for 568 gait samples formed by dividing a total of 2295 steps into four consecutive steps. In FIG. 11, it can be seen that the recognition rate is increased as the dimension of the feature space increases, and that a maximum recognition rate of 91.3% is exhibited in a 12-dimensional feature space.

It is confirmed that since a wearable device is used unlike user recognition methods using video data-based step analysis, the method according to the present invention is an effective method in that restrictions on a data measurement environment are low and high recognition performance can be obtained from a low-capacity acceleration pattern.

The apparatus and the method for gait type classification using a pressure sensor of a smart insole according to the present invention have the following effects.

First, it is possible to classify types of pieces of gait data in which various variances are present using only the pressure sensor.

Second, a section corresponding to one step is defined in all the pieces of gait data by detecting a swing phase which is a state in which a foot floats in the air, and the pieces of gait data for each unit step are divided, thereby improving classification accuracy.

Third, variances of the pieces of gait data due to a gait speed are removed, data resizing is performed for equal comparison between the pieces of unit gait data, and normalization is performed on the pieces of unit gait data, thereby equalizing lengths of the pieces of divided data such that type classification of the pieces of unit gait data in which various variances are present can be accurately performed.

Fourth, it is possible to effectively extract discriminant features from the pieces of normalized unit gait data using the Null-LDA method when the discriminant features are extracted for the gait type classification.

As described above, it will be understood that the present invention can be implemented in a modified form without departing from the essential characteristics of the present invention.

Therefore, it should be construed that the above-described embodiments are to be considered in an illustrative point of view rather than a restrictive point of view, that the scope of the present invention is defined by the appended claims rather than by the foregoing description, and that all differences within an equivalent scope of the claims fall within the present invention.

What is claimed is:

1. An apparatus for gait type classification, comprising:
a gait data measuring part configured to measure pieces of gait data using a pressure sensor;
a pre-processor configured to define a section of a unit step in all the pieces of gait data, divide the pieces of gait data for each unit step, and normalize the pieces of divided gait data to equalize lengths of the pieces of divided gait data;
a feature extractor configured to extract features suitable for gait type classification from the pieces of pre-processed gait data; and
a gait type classifier configured to receive the extracted features and determine and classify a final gait type.

2. The apparatus of claim 1, wherein:
a plurality of pressure sensors of the gait data measuring part are provided at spaced positions in an insole of a shoe; and
according to pressure strength, each of the plurality of pressure sensors discriminate, measure, and store a state in which a foot is separated from the ground and a pressure is absent as “0,” a state in which a weak pressure is present as “1,” and a state in which a strong pressure is present as “2.”

3. The apparatus of claim 1, wherein the pre-processor classifies one gait cycle into a swing phase which is a state in which one foot floats in the air and a stance phase which is a state in which one foot is in contact with the ground, detects the swing phase, and constitutes a gait sample per unit step from all the pieces of measured gait data on the basis of the detected swing phase.

4. The apparatus of claim 3, wherein, since a foot is separated from the ground at a sampling point in the swing phase, criteria for detecting the swing phase and the stance phase, where all utilized values of the plurality of pressure sensors should be 0, are defined as follows:
$$\{ \begin{matrix}SWING if p = 0 \\ STANCE if p > 0\end{matrix} \\ p = {\sum }_{i = 1}^{8} ( value of \text{i} \text{-} \text{th} pressure sensor ) ,$$
wherein p denotes the sum of all the values of the plurality of pressure sensors at a certain sampling point.

5. The apparatus of claim 3, wherein, during the gait, the swing phase of the left foot is detected, and on the basis of the detected swing phase of the left foot, a timing from a start point of the swing phase of the left foot to an end point of the stance phase is defined as one piece of step data.

6. The apparatus of claim 1, wherein the pre-processor removes sections each having a length of 0.5 seconds or less among sections for one step by regarding such sections as being generated by a false positive (FP) swing phase.

7. The apparatus of claim 6, wherein the pre-processor measures a shortest time among the pieces of noise-removed unit step data, resizes each of the pieces of unit step data to have a length corresponding to a measured unit time, and normalizes the sections for one step to have the same size.

8. The apparatus of claim 1, wherein the feature extractor extracts discriminant features from the pieces of normalized unit gait data using a null-space linear discriminant analysis (Null-LDA) method when the discriminant features are extracted for gait type classification.

9. The apparatus of claim 8, wherein the Null-LDA method is a method in which a within-class scatter matrix SW and a between-class scatter matrix SB of N pieces of learning data xₖ constituted with C classes are respectively defined as
$${S}_{W} = {{\sum }^{C}}_{i = 1} {\sum }_{{x}_{k} ∈ {c}_{i}} ( {x}_{k} - {μ}_{i} ) {( {x}_{k} - {μ}_{i} )}^{T} and$$
$${S}_{B} = \frac{1}{N} {\sum }_{i = 1}^{C} {N}_{i} ( {μ}_{i} - μ ) {( {μ}_{i} - μ )}^{T} ,$$
and an objective function is set as
$${W}_{LDA} = {argmax}_{W} \frac{\vert {W}^{T} {S}_{B} W \vert}{\vert {W}^{T} {S}_{W} W \vert}$$
to maximize a ratio of a variance of the within-class scatter matrix SW to a variance between averages of the between-class scatter matrix SB.

10. The apparatus of claim 9, wherein W_{LDA} satisfying the objective function is calculated by eigenvalue analysis of S_{W}⁻¹S_{B}, and a feature yₖ of a sample xₖ is calculated as W_{LDA}^{T}xₖ using WLDA.

11. The apparatus of claim 9, wherein, when a small sample size (SSS) problem, in which the number of samples is smaller than the number of dimensions of pieces of data, because S_{W}⁻¹ is not present, and thus a solution is not calculated, occurs, a kernel principal component analysis (PCA)+LDA method, in which the PCA is applied to decrease the number of dimensions of pieces of data to less than that of the scatter matrix SW and then an LDA method is applied, is used, and the Null-LDA method of projecting a within-class data into a null space and then searching a subspace where a scatter matrix is maximized is applied.

12. The apparatus of claim 11, wherein, when a total scatter matrix ST is defined as
$${S}_{T} = {\sum }_{i = 1}^{C} {\sum }_{{x}_{k} ∈ {c}_{i}} ( {x}_{k} - μ ) {( {x}_{k} - μ )}^{T} ,$$
a projective matrix of the PCA+LDA method is W_{PCA+LDA}=W_{LDA}^{T}W_{PCA}^{T}, wherein W_{PCA}=argmax_{W}|W^{T}S_{T}W| and
$${W}_{LDA} = arg {max}_{W} \frac{\vert {W}^{T} {W}_{PCA}^{T} {S}_{B} {W}_{PCA} W \vert}{\vert {W}^{T} {W}_{PCA}^{T} {S}_{W} {W}_{PCA} W \vert} ,$$
and the Null-LDA method calculates a projective matrix W_{NLDA} satisfying the objective function of W_{NLDA}=argmax_{|W}_{^{T}}_{S}_{_{W}}_{W|=0}|W^{T}S_{B}W| in a space in which |W^{T}S_{W}W|=0 and |W^{T}S_{B}W|≠0 using a null space of the within-class scatter matrix SW.

13. The apparatus of claim 8, wherein:
the gait data measuring part further includes an accelerometer sensor provided in an insole of a shoe;
the feature extractor extracts the features suitable for user identification from the pieces of pre-processed data; and
wherein the apparatus further includes a user identifier configured to receive the features suitable for the user identification extracted by the feature extractor and perform the user identification.

14. The apparatus of claim 13, wherein:
the Null-LDA method defines a between-class scatter matrix SB and a within-class scatter matrix SW for N samples constituted with C classes as
$${S}_{B} = \frac{1}{N} {\sum }_{i = 1}^{C} {N}_{i} ( {μ}_{i} - μ ) {( {μ}_{i} - μ )}^{T} and$$
$${S}_{W} = {{\sum }^{C}}_{i = 1} {\sum }_{{x}_{m} ∈ {c}_{i}} ( {x}_{m} - {μ}_{i} ) {( {x}_{m} - {μ}_{i} )}^{T} ,$$
respectively, wherein xₘϵR^{n×1} is an mth sample belonging to a class Ci, and μ and μi refer to an average of all samples and an average of samples belonging to class Ci, respectively.

15. The apparatus of claim 14, wherein:
the Null-LDA method projects the samples into the null space of the within-class scatter matrix SW so as to maximize a discriminant power between classes and calculates projective vectors satisfying the objective function using W_{NLDA}=argmax_{|W}_{^{T}}_{S}_{_{W}}_{W|=0}|W^{T}S_{B}W| so as to maximize a variance between averages of the classes; and
W_{NLDA} is a projective matrix constituted with n′ projective vectors wₜ, t=1, . . . , n′.

16. The apparatus of claim 15, wherein a feature vector y for a sample x is y=W^{T}x (ϵR^{n×1}).

17. A method for gait type classification, comprising:
measuring pieces of gait data using a plurality of pressure sensors provided at spaced positions in an insole of a shoe;
removing sections each having a length less than a preset time among sections for one step by regarding the sections each having a length less than the preset time as being generated by a false positive (FP) swing phase;
pre-processing the pieces of gait data by measuring a shortest time among the pieces of noise-removed unit step data, resizing each of the pieces of unit step data to a length corresponding to a measured unit time, normalizing sections for one step to have the same size; and
extracting features suitable for gait type classification from the pieces of pre-processed data, receiving the extracted features as an input, and determining and classifying a final gait type.

18. The method of claim 17, wherein the measuring of the pieces of gait data includes classifying, measuring, and storing, according to pressure strength, a state in which a foot is separated from the ground and a pressure is absent as “0,” a state in which a weak pressure is present as “1,” and a state in which a strong pressure is present as “2.”

19. The method of claim 17, wherein the pre-processing of the pieces of gait data includes:
classifying one gait cycle into a swing phase in a state in which one foot is floating in the air and a stance phase in a state in which one foot is in contact with the ground; and
detecting the swing phase and constituting a gait sample per unit step from all the pieces of measured gait step data on the basis of the detected swing phase.

20. The method of claim 19, wherein, since a foot is separated from the ground at a sampling point in the swing phase, all utilized values of the plurality of pressure sensors should be 0, criteria for detecting the swing phase and the stance phase are defined as
$$\{ \begin{matrix}SWING if p = 0 \\ STANCE if p > 0\end{matrix} \\ p = {\sum }_{i = 1}^{8} ( value of \text{i} \text{-} \text{th} pressure sensor ) ;$$
and
p denotes the sum of all the values of the plurality of pressure sensors at a certain sampling point.

21. The method of claim 19, further comprising:
during the gait, detecting the swing phase of a left foot; and
on the basis of the detected swing phase of the left foot, defining a timing from a start point of the swing phase of the left foot to an end point of the stance phase as one piece of step data.
